SEQUENCE LISTING (12 sequences)

SEQ ID NO: 1 - DNA, 20 bp, artificial sequence
  Forward primer for RT-PCR on DRD2 cDNA template
ctgctcatcgctgtcatcgt

SEQ ID NO: 2 - DNA, 16 bp, artificial sequence
  Reverse primer for RT-PCR on DRD2 cDNA template
ctcgcgggacacagcc

SEQ ID NO: 3 - DNA, 21 bp, artificial sequence
  TaqMan Probe for RT-PCR on DRD2 cDNA template
tcggcaacgtgctggtgtgca

SEQ ID NO: 4 - DNA, 22 bp, artificial sequence
  Forward primer for RT-PCR on Kir2.3 cDNA template
cttcccccactgactcttcaag

SEQ ID NO: 5 - DNA, 19 bp, artificial sequence
  Reverse primer for RT-PCR on Kir2.3 cDNA template
ccagcagtccagccacctt

SEQ ID NO: 6 - DNA, 26 bp, artificial sequence
  TaqMan Probe for RT-PCR on Kir2.3 cDNA template
tgccctctttgctctcagaaccttgg

SEQ ID NO: 7 - DNA, 15 bp, artificial sequence
  Forward primer for RT-PCR on Human beta actin (ACTB) cDNA template
tcaccgagcgcggct

SEQ ID NO: 8 - DNA, 21 bp, artificial sequence
  Reverse primer for RT-PCR on Human beta actin (ACTB) cDNA template
taatgtcacgcacgatttccc

SEQ ID NO: 9 - DNA, 21 bp, artificial sequence
  TaqMan Probe for RT-PCR on Human beta actin (ACTB) cDNA template
cagcttcaccaccacggccga

SEQ ID NO: 10 - DNA, 21 bp, artificial sequence
  Forward primer for RT-PCR on HPRT cDNA template
tgacactggcaaaacaatgca

SEQ ID NO: 11 - DNA, 20 bp, artificial sequence
  Reverse primer for RT-PCR on HPRT cDNA template
gcttgcgaccttgaccatct

SEQ ID NO: 12 - DNA, 32 bp, artificial sequence
  TaqMan Probe for RT-PCR on HPRT cDNA template
ctttgctttccttggtcaggcagtataatcca

(19) United States
(12) Patent Application Publication
(10) Pub. No.: US 2008/0274455 A1
(43) Pub. Date: Nov. 6, 2008

(54) Use Of Genes As Molecular Markers In Diagnosis Of Schizophrenia And Diagnostic Kit For The Same

(76) Inventors: Laszlo Puskas, Szeged (HU); Agnes Zvara, Szeged (HU); Miklos Santha, Szeged (HU); Gyorgy Szekeres, Szeged (HU); Zoltan Janka, Szeged (HU)

Correspondence Address: HAHN & VOIGHT PLLC, 1012 14TH STREET, NW, SUITE 620, WASHINGTON, DC, 20005, US

(21) Appl. No.: 11/568,467

(22) PCT Filed: May 2, 2005
(86) PCT No.: PCT/HU05/00044

§ 371 Date: Jan. 8, 2007

(30) Foreign Application Priority Data
Apr. 30, 2004 (HU) P 0400904

(51) Int. Cl. C12Q 1/68; C40B 30/04; C12Q 1/02; G01N 33/53; C07H 21/04

(52) U.S. Cl. 435/6; 506/9; 435/29; 435/7.8; 536/24.31

(57) ABSTRACT

Drug-naive and drug-free schizophrenic PBL were screened to identify additional markers that are differentially expressed compared to healthy individuals using microarray and quantitative real-time PCR (QRT-PCR) techniques. Genes for dopamine D₂ receptor (DRD2) and inwardly rectifying potassium channel (Kir2.3) were found to be overexpressed in microarray analysis. Increased mRNA levels were confirmed by QRT-PCR using SybrGreen method and dual labeled TaqMan probes.

The invention relates to a method for diagnosing schizophrenia in a subject comprising assessing the level or the expression level of at least one of the following genes or proteins: Kir2.3 or DRD2 or a gene encoding Kir2.3 or DRD2. The invention further relates to agents and uses thereof, said agents specifically binding to said proteins or nucleic acids encoding them, diagnostic kits and screening methods.

Use of both molecular markers allow prediction of schizophrenia and help to follow efficiency of drugs in therapy in order to provide a more tailored medication for schizophrenic patients.

The invention relates to the field of diagnosing schizophrenia.

In particular, the invention relates to a method for diagnosing schizophrenia in a subject comprising assessing the level or the expression level of at least one of the following genes or proteins: Kir2.3 or DRD2 or a gene encoding Kir2.3 or DRD2. The invention further relates to agents and uses thereof, said agents specifically binding to said proteins or nucleic acids encoding them, diagnostic kits and screening methods.

### BACKGROUND ART

Schizophrenia is a severe neuropsychiatric disorder characterized by disturbances in mental, emotional and motor processes⁵ This complex and devastating disease affects approximately 1% of the human population.

There is an abundance of data evidencing that there is a genetic linkage of schizophrenia and that expression pattern of certain genes is altered in patients with schizophrenia. Intensive studies have been carried out to find appropriate genetic markers allowing diagnosis of schizophrenia either before development of symptoms or with an increased reliability. In general, many genes or proteins said to be a marker in schizophrenia were and, in part, are still poorly characterized or there is no convincing evidence that there is significantly different level of expression between healthy and affected individuals.

In WO 0144269 (Johnston-Wilson et al.) brain protein markers for various psychiatric and neurological disorders, including schizophrenia, are taught. Said marker proteins have been identified by 2-D (two dimensional) gel electrophoresis.

In EP 1 348963 A1 (Cochran et al.) it is described that the gene of the voltage-gated potassium channel 3.1 (Kv3.1) α-subunit shows altered expression in schizophrenia and diagnostic and screening methods, based on this finding, are disclosed. To present inventor's knowledge this is the only report on the relation of a potassium channel protein and schizophrenia.

A useful animal model for studying Kir2.3 is described in WO02400661, wherein transgenic mice containing Kir2.3 potassium channel gene disruptions are described. However, no specific mention of use of said mice for studying schizoprenia was found in WO02400661.

One of the hypotheses explaining the nature of the disease is based on dysfunction of dopaminergic system, especially an increased occupancy of D2 subclass of dopamine receptors (DR) by dopamine in schizophrenic patients.⁶ Today antipsychotic drugs used in treatment of schizophrenia act as antagonists of D2 subclass of DRs.⁷ In U.S. Pat. No. 4,931,270 (Horn et al.) methods and labelled compounds are provided to detect altered distribution of dopamine D2 receptors in schizophrenia.

Expression of neurotransmitters, neuropeptides and D2 subclass of dopamine receptor genes (D₂, D₃, D₄ subtypes) in immune cells, mainly in peripheral blood lymphocytes (PBL), has been reported earlier by several authors.^{1,4,8,9,10} It has been suggested, that they may reflect the status of corresponding brain receptors. In recent years, D₃ subtypes of D2 subclass of dopamine receptor genes (DRD3) were found to be overexpressed in schizophrenic PBL and proposed to be a useful peripheral marker for identification and follow-up of schizophrenia.^{3,11} The same laboratory reported that α7 nicotinic acetylcholine receptor (α7AChR) gene was also differentially expressed in schizophrenic patients, since in affected PBL a decreased mRNA level of α7AChR had been detected.¹² Since dopamine interacts directly with DRD3 and DRD2 on T cells and activate integrin mediated T cell adhesion, overexpression of these genes in T cells can serve not only as a passive diagnostic marker but can reflect a dynamic functional interaction.¹³

In recent years fast increase of sequence data and new molecular methods developed to analyze gene expression profile either in high throughput format (microarray technology) or by more precise methods (e.g. quantitative real-time PCR) promise an effective search for new markers.

In EP 1 132 483 A2 (Nawa et al.) a DNA micro-array is used as a tool for finding proteins differentially expressed in schizophrenia. Though a large number of proteins are assumed to be appropriate markers, no convincing evidence is shown in the description.

In WO 0136473 (Vogeli et al.), an extensive study of a large number of putative G-protein coupled receptors is described. Upon expression analysis, a few of them, identified only by their sequence, were proposed to be related to schizophrenia. However, no direct evidence appears to have been provided.

A microarray approach has been utilized in the finding of a decreased expression of G-protein signalling 4 (RGS4) in patients of schizophrenia, as taught in WO 0216653.

Vawter et al. screened the PBLs of five individual family members with schizophrenia with a comparison of unaffected individuals to identify differentially expressed genes using microarray of 1128 brain related genes but neither DRD2 nor Kir2.3 were presented on that microarray.³⁵

In WO2004/082570 the use of DRD2 in a number of neurological diseases in mentioned. However, no specific suggestion can be found in relation to schizophrenia.

Though a number of possibly suitable marker genes are known in the prior art, there is still a need to identify further genes which are applicable in the diagnosis of schizophrenia or, if used in combination with known markers, enhance the reliability of existing methods.

Despite extensive studies no suggestion could be found in the prior art that a gene of an inwardly rectifying potassium channel might be a marker of schizophrenia.

Moreover, to the best of inventor's knowledge, in the prior art the use of the gene of DRD2 as a specific diagnostic marker in schizophrenia has not been not suggested.

Moreover, it has not been known that said genes or the proteins encoded by them, could be peripheral molecular markers in schizophrenia.

The object of the present invention is to provide further diagnostic markers useful in methods for diagnosing schizophrenia.

Present inventors applied both high throughput microarray analysis to pre-scan, and quantitative real-time PCR technique to identify multiple genes expressed differentially in schizophrenic PBL. Genes for DRD2 and Kir2.3 were found to be overexpressed in microarray study. Two genes from DNA microarray results were confirmed by using SybrGreen and TaqMan probes in quantitative real-time PCR. Since neither affected nor non-affected individuals were under antipsychotic or other medication treatment, elevated mRNA levels of DRD2 and Kir2.3 reflect the disorder itself, not the effect of medication. Combination of multiple peripheral molecular genetic markers would be extremely helpful for precise early diagnosis, follow-up of schizophrenia and in decision of effective therapy.

### BRIEF DESCRIPTION OF THE INVENTION

The invention relates to a method for diagnosing schizophrenia in a subject comprising
assessing the expression level of at least one of the following genes:
   a gene encoding Kir2.3 (inwardly rectifying potassium channel 2.3) and/or
   a gene encoding DRD2 (dopamine D2 receptor)

in a biological sample taken from said subject and
comparing the expression level in the biological sample to a baseline expression level(s) of the respective gene(s) typical of healthy subjects or in a healthy subject or to a predetermined control baseline expression level,

wherein a higher gene expression level in the biological sample is considered as an indication of the fact that the subject suffers in or is susceptible to schizophrenia.

Preferably, the method of the invention comprises assessing the expression level of at least a gene encoding Kir2.3. More preferably, level of a gene encoding DRD2 is also assessed.

In a preferred embodiment, the method of D1 wherein assessment of the expression level comprises hybridization of a hybridization probe to a nucleic acid obtained from the biological sample, said nucleic having a sequence
of a gene encoding Kir2.3 and/or
of a gene encoding DRD2 or
a part thereof
or a sequence complementer to any of the above.

Preferably, in the method of the invention a hybridization probe is hybridized to at least a nucleic acid having the a sequence of a gene encoding Kir2.3. In a preferred embodiment a further probe is hybridized to a nucleic acid having the a sequence of a gene encoding DRD2.

Preferably, the nucleic acid obtained from the biological sample is mRNA or cDNA.

In a preferred embodiment, the expression level is determined by microarray analysis.

Preferably, the expression level is determined by a PCR method, preferably a real time PCR method, more preferably a quantitative real time PCR method.

The biological sample is taken from brain, spinal cord, lymphatic fluid, lymphatic organ, bone marrow, blood, urine or feces.

In a preferred embodiment the biological sample comprises immune cells. Preferably, the biological sample comprises peripheral blood cells, preferably lymphocites, and the gene expression level is assessed in these cells.

In a further embodiment, the invention relates to a method for diagnosing schizophrenia in a subject comprising
assessing the level of at least one of the following proteins:
   Kir2.3 and/or
   DRD2

in a biological sample taken from said subject and
comparing the level of the protein(s) in the biological sample to a baseline level value of the respective protein typical in healthy subjects or in a healthy subject or to a predetermined control baseline level,

wherein a higher level in the biological sample is considered as an indication of the fact that the subject suffers in or is susceptible to schizophrenia.

Preferably, at least the level of Kir2.3 is assessed. More preferably, at least the level of both Kir2.3 and DRD2 are assessed.

In a preferred embodiment, assessing the level of the protein comprises determination of the activity or the concentration of the protein.

Preferably, the biological sample is taken from tissues or body fluids as defined above. Highly preferably, the biological sample isolated from the subject comprises immune cells, even more preferably the biological sample isolated from the subject comprises peripheral blood cells, preferably lymphocites, and the activity and/or concentration of the protein(s) is assessed in these cells.

In a further aspect the invention relates to a use of an agent capable of specifically binding to at least one of the following:
Kir2.3 or a nucleic acid encoding the same and/or
   DRD2 or a nucleic acid encoding the same

for diagnosing schizophrenia.

Preferably, the agent is capable of specifically binding to at least Kir2.3 or a nucleic acid encoding the same. More preferably, both an agent capable of specifically binding to Kir2.3 or a nucleic acid encoding it and an agent capable of specifically binding DRD2 or a nucleic acid encoding it are used.

In a preferred embodiment, the agent is an oligonucleotide specifically hybridizing to any of the nucleic acids encoding DRD2 or Kir2.3 or a complementer or a part thereof. The oligonucleotide is preferably a probe having a label. The label is preferably a fluorescent label or a radioactive label. Highly preferably, the probe comprises a fluorescent label and a quencher.

In a preferred embodiment the agent is an oligonucleotide primer useful in PCR.

In a further preferred embodiment the agent is a molecule specifically binding to at least one of the following:
Kir2.3 and/or
DRD2.

Preferably, the agent is an antibody, more preferably a monoclonal antibody.

In a further aspect the invention relates to a diagnostic kit for diagnosing schizophrenia in a subject comprising

at least one agent capable of specifically binding to at least one of the following:
Kir2.3 or a nucleic acid encoding the same, and/or
DRD2 or a nucleic acid encoding the same and

means for producing a detectable signal upon specific binding of the agent.

Preferably, the kit comprises at least an agent capable of specifically binding to Kir2.3 or a nucleic acid encoding it. In a preferred embodiment, the kit also comprises an agent capable of specifically binding to DRD2 or a nucleic acid encoding it.

In a preferred embodiment, the means producing a detectable signal comprises a label, preferably an enzymatic label, a fluorescent label or a radioactive label.

In a preferred embodiment, the agent is a probe comprising an oligonucleotide specifically hybridizing to any of the nucleic acids or a complementer or a part thereof. The probe may comprise an oligonucleotide and a label bound thereto. In a preferred embodiment, the label is a fluorescent label, and preferably the probe comprises a fluorescent label and a quencher.

In a further embodiment the probe is an oligonucleotide and the label is an intercalating agent, e.g. SYBR-green or ethidium bromide.

Preferably, the diagnostic kit further comprises at least one oligonucleotide primer pair useful in amplifying at least one of the nucleic acids.

In a preferred embodiment, in the diagnostic kit of the invention the agent is a molecule specifically binding to at least one of the following proteins:
Kir2.3 and/or
DRD2.

Preferably, the agent is an antibody, preferably a monoclonal antibody.

In the kit the means for detection of the specific binding can be a label bound to the antibody or a further labelled antibody capable of binding to the first antibody.

In a further aspect the invention relates to an oligonucleotide probe for diagnosing schizophrenia in a subject said probe comprising and oligonucleotide capable of specifically hybridizing to any of the nucleic acids encoding
Kir2.3 and/or DRD2

or a complementer or a part thereof.

Preferably the probe according to the invention comprises a fluorescent label, and preferably a quencher, bound to the oligonucleotide.

In a further aspect the invention relates to a method for screening for a potential therapeutic agent that modulates symptoms of schizophrenia comprising contacting a candidate compound with at least one of the following proteins or nucleic acids:
Kir2.3 or a nucleic acid encoding it and/or
DRD2 or a nucleic acid encoding it

and assessing a the effect of the compound,

wherein a compound effecting the function of any of the said nucleic acids or proteins is considered as a potential therapeutic agent that modulates symptoms of schizophrenia.

Preferably, in the method a candidate compound is contacted at least with Kir2.3 or a nucleic acid encoding it. In a preferred embodiment the candidate compound is also contacted with DRD2 or a nucleic acid encoding it.

Preferably, the candidate compound is contacted with at least one of the following:
Kir2.3 and/or
DRD2,

and assessing the effect of the compound on the activity of the respective protein,

wherein a compound decreasing the activity is considered as a potential therapeutic agent in schizophrenia.

According to a preferred method the candidate compound is contacted with at least one of the following:
a nucleic acid encoding Kir2.3 and/or
a nucleic acid encoding DRD2

in a cell expressing said nucleic acid(s),

and assessing and evaluating the effect of the compound on the expression level of said nucleic acid(s),

wherein a compound decreasing the expression level is evaluated as a potential therapeutic agent in schizophrenia.

In a further embodiment the candidate compound is administered to an animal model of schizophrenia overexpressing at least one of the following:
Kir2.3 or a nucleic acid encoding it and/or
DRD2 or a nucleic acid encoding it

and assessing the effect of the compound on symptoms associated with schizophrenia.

Preferably, in the animal model at least Kir2.3 or a gene encoding it is overexpressed. More preferably, both Kir2.3 and DRD2, or genes encoding them are overexpressed.

### DEFINITIONS

A “subject” is an individual person or animal. A subject may be a patient who has or may have or is suspected to have a given disorder, or may be a healthy person or animal who optionally can be a control.

A “biological sample” includes any body fluid or tissue obtained from a subject and optionally processed or digested or prepared for study, and containing a protein marker and/or nucleic acid(s) encoding the marker and/or substrates for the protein and/or products produced by the marker. Moreover, a biological sample may be a cell culture, e.g. a primary cell culture obtained by culturing cells of the patient.

A “nucleic acid obtained from a biological sample” is a given nucleic acid which is a component of the sample but which is made available for further studies, e.g. by processing the sample, e.g. by purifying it to certain extent, or by isolating said nucleic acid.

An “immune cell” is a cell which is a descendent of a bone marrow stem cell. Preferred immune cells are cells of myeloid or lymphoid origin. Immune cells are e.g. lymphocytes, natural killer (NK) cells, leukocytes, granulocytes, monocytes, e.g. phagocytes etc.

“Isolation” of a given material means changing its original environment by Man.

A “probe” is a compound capable of reacting with or binding to a biological molecule to be studied, i.e. to the target molecule. Preferably, a probe is designed to contribute to the emission of signals upon reaction with or binding to the said biological molecule.

Preferred type probes for detection of nucleic acids are probes comprising an oligonucleotide (oligonucleotide probes), e.g. fluorescent probes. A highly preferred type of probes contain a fluorescent dye and a quencher capable of fluorescence resonance energy transfer if the probe is not bound to the target molecule (e.g. TaqMan type probes). Special types of such probes are e.g. molecular beacons.

The “expression level” of a gene relates to any measure of the expression allowing quantitative or semi-quantitative assessment or estimation thereof suitable for comparison of expression levels in various samples or subjects. Similarly, the “level” of a protein relates to any measure of the concentration, quantity, effect or activity of the protein allowing quantitative or semi-quantitative assessment or estimation thereof suitable for comparison of levels in various samples or subjects.

“Specific hybridization” relates to binding of a poly or oligonucleotide to an other due to sequence similarity under defined conditions. In a preferred embodiment stringent conditions are applied. Under stringent condition any stringent conditions defined in usual manuals or textbooks in respect of hybridization of probes can be applied. For example, hybridization conditions as defined in the Examples can be applied. Alternatively, As an example, stringent conditions are e.g. 65° C. in a buffer containing 1 mM EDTA, 0.5 M NaHPO₄, (pH 7.2), 7% (w/v) SDS. Preferably said binding is detectable e.g. by usual means for detecting double stranded DNA.

“DRD2” (dopamine D2 receptor) relates to the D2 subtype of the dopamine receptor. This G-protein coupled receptor inhibits adenyl cyclase activity.

Amino acid sequence of DRD2 protein is given e.g. in PubMed Protein Database at the following Entries: BC021195.2, P14416, NP_{—}057658

Nucleotide sequence of a nucleic acid (e.g. mRNA or cDNA) is given e.g. in PubMed Nucleic Acid Database at the following Entries:X51645, M30625 and NM016574.

Moreover, examples for DRD2 receptor protein and nucleic acids coding therefore are given in the following publications: Strausberg, R. L. et al (2002)³⁶, Araki, K. at al. (1992)³⁷, Dearry, A. (1991)³⁸, Monsma, F. J. (1989)³⁹, Dal Toso, R. (1989)⁴⁰, Grandy, D. K. (1989)⁴¹ and in WO 2004/082570.

“Kir2.3” (inwardly rectifying potassium channel 2.3) is an integral membrane protein controlled by G proteins and a member of inwardly rectifying K+ channels, which have a greater tendency to allow potassium to flow into the cell rather than out of it. This asymmetry in potassium ion conductance plays a key role in the excitability of muscle cells and neurons. Voltage dependence of Kir2.3 is regulated by the concentration of extracellular potassium; as external potassium is raised, the voltage range of the channel opening shifts to more positive voltages.

Examples for amino acid sequence of the protein are given in Entrez Protein Database e.g. at the following Entries: P52190, NP_{—}690607, NP_{—}004972, Q64198, P52189, P48050, AAG17049, AAG53738

Examples for nucleic acid sequences encoding Kir2.3 are given in Entrez Nucleotides Database e.g. at the following Entries: mRNA: NM_{—}152868, NM_{—}004981 (human); cDNS: AF187874, AF183917 (domestic guinea pig; Cavia porcellus)

Moreover, examples for Kir2.3 receptor and nucleic acids coding therefor are given in the following publications: Perier, F. (1994)⁴², Tang, W. (1994)⁴³, Makhina, E. N. (1994)⁴⁴, Budarf, M. L. (1995)⁴⁵.

If a “part” of a protein or of a nucleic acid encoding it or of a gene is mentioned herein it is understood that said part has an essential feature of the said protein or nucleic acid or gene allowing to obtain the same result as if the whole molecule would be used. For example, a part of a protein has the same function or, if the detection of said protein is mentioned, a part of the protein can be detected by the same means. For example, it has the same structural element, e.g. a domain or an epitope characteristic to the whole protein.

If a part of a nucleic acid or a gene is mentioned it must have a sufficient length so as to have a sequence motif unambiguously specific to the whole nucleic acid or gene. For example, under stringent conditions, the same probe can be hybridized to the whole molecule as to the appropriate part thereof.

### BRIEF DESCRIPTION OF THE FIGURES

FIG. 1 shows the variation of relative expression level of DRD2 and Kir2.3 genes in non-affected control samples. Relative expression levels are plotted as log₂ ratios on Y-axis. Dashed bars and white bars represent Kir2.3 gene expression levels analyzed by SybrGreen and TaqMan protocols, respectively. Grey and black bars represent the DRD2 gene expression level analyzed by SybrGreen and TaqMan protocols, respectively. (In the figure the meaning of MC is male control; the meaning of FC is female control.

On FIG. 2 the relative expression levels of DRD2 (A) and Kir2.3 (B) genes in schizophrenic samples are indicated. Ratios are shown as log₂ ratios on Y-axis. White and black bars represent data analyzed by QRT-PCR using SybrGreen dye and TaqMan protocols, respectively. Grey bars show the relative expression level of DRD2 and Kir2.3 gene analyzed by microarray method. (A) White dotted line at (log₂ratio=1.4) and black dashed line at (log₂ratio=1.3) show the background expression level of DRD2. (B) White dotted line at (log₂ratio=1.8), and black dashed line at (log₂ratio=1.5) show the background expression level of Kir2.3. Calculation the value of background relative expression level is based on the variation of relative expression level of DRD2 and Kir2.3 genes in non-affected control samples. (M, male patient; F, female patient).

FIG. 3. Hierarchical clustering based on expression dataset clearly separates into two main clusters: the non-affected control and affected samples with the evident exception of schizophrenic sample F5. (MC, male control; FC, female control; M, male patient; F, female patient)

### DETAILED DESCRIPTION OF THE INVENTION

Below certain embodiments of the invention are illustrated in more detail by way of examples. It is to be understood that the scope of the invention is not restricted to these examples.

The invention pertains to novel biochemical methods for diagnosing schizophrenia and is based on the finding that genes encoding the dopamine D2 receptor DRD2 and the inwardly rectifying potassium channel Kir2.3 are upregulated in patients of schizophrenia.

DRD2 belongs to the D2 subclass of DRs and is coupled to G-protein with an inhibiting effect of adenyl cyclase activity.²⁷ Receptor-activated G-proteins can either activate (Kir3.1) or inactivate inwardly rectifying potassium channels (Kir2.3).^{28,29} Several different potassium channels are known to be involved in electrical signaling in the nervous system. Kir2.3 is a member of Kir2 family of constitutively active inwardly rectifier K⁺ channels carrying large inward and small outward potassium current.³⁰ This asymmetry in potassium ion conductance plays a key role in the excitability of muscle cells and neurons. DA (dopamine) through D2 subclass of DRs directly activates an IAP (Islet activating protein, pertussis toxin)-sensitive G protein coupled with inward rectifier potassium channels in rat substantia nigra neurones.³¹ Lavine et al. demonstrated that DRD2 and another inwardly rectifying potassium channel Kir3, form a stable functional complex both in vitro and in vivo.³² Though the present invention is by no means to be limited by any theory behind, these results suggest a strong association between DRs and inwardly rectifying potassium channels.

In one embodiment of the invention the expression level of either the gene encoding DRD2 or the gene encoding Kir2.3 or both is assessed in a biological sample taken from a subject. The expression level determined in the biological sample is compared to a baseline expression level of the respective gene, typical of healthy subjects. If the gene expression level in the biological sample is higher than the baseline level, this finding is considered as an indication of the fact that the subject suffers in or is susceptible to schizophrenia.

In an other embodiment the level of either a DRD2 protein or a Kir2.3 protein is assessed in a biological sample taken from a subject. The level of the protein(s) in the biological sample is compared to a baseline level value of the respective protein typical in healthy subjects. If the level in the biological sample is higher than the baseline level the finding is considered as an indication of the fact that the subject suffers in or is susceptible to schizophrenia.

It is to be emphasized that other markers of schizophrenia have been known in the art, examples for which are listed in the introductory part of the present description. The present novel markers also can be used together with the known markers improving the reliability of diagnosis.

The baseline expression level can be the expression level of the same gene in a healthy, i.e. non-schizophrenic, subject. More preferably the baseline level is defined as an average of expression levels of a reference group of healthy subjects. It may be advisable if the reference group is selected carefully an the subjects in this group and the subject to be diagnosed advantageously match each other in respect of certain characteristics, e.g. race, age, sex etc, though our results suggest that variations within healthy subjects are usually smaller than the differences between healthy and schizophrenic subjects. The baseline level is expediently defined relative to a suitable control, e.g. an internal reference control. Alternatively, for a more general use the reference group of subjects should be sufficiently diverse in terms of age, sex, social status, geographical distribution, previous drug and medical histories, etc. and of sufficient size to provide a meaningful statistical value. In this way expression of the diagnostic marker genes or proteins of the invention is measured in the set of samples representing the “normal” population. Baseline level value is calculated from a statistic of these results.

Biological samples can be taken from any tissue showing differential expression of the marker genes or proteins of the invention. Such tissues can be, though not exclusively and not under any circumstances, brain, spinal cord, lymphatic fluid, blood etc. Methods for obtaining sample and preparing them for measurements are well known in the art.

The skilled person has known for a long time a number of methods for quantitative detection of proteins levels or of gene expression levels in a sample. All these methods may be applicable in the invention.

In one embodiment of such a detection scheme, a cDNA molecule is synthesized from an RNA molecule of interest (e.g. by reverse transcription of the RNA molecule into cDNA). A sequence within the cDNA is then used as the template for a nucleic acid amplification reaction, such as a PCR amplification reaction, or the like. The nucleic acid reagents used as primers in the reverse transcription and nucleic acid amplification steps of this method are chosen from DRD2 and Kir2.3 gene nucleic acid reagents. Those skilled in the art are familiar with techniques for designing and obtaining suitable primers (see the Examples).

Additionally, it is possible to perform such gene expression assays “in situ”, i.e. directly upon tissue sections (fixed and/or frozen) of patient tissue obtained from biopsies or resections, such that no nucleic acid purification is necessary. Nucleic acid reagents may be used as probes and/or primers for such in situ procedures. Alternatively, if a sufficient quantity of the appropriate cells can be obtained, standard Northern analysis can be performed to determine the level of mRNA expression of the marker genes.

However, recently, certain relatively new method have gained widespread applicability.

As demonstrated by the Examples below, one technique for establishing baseline levels may involve real time quantitative PCR (RT-PCR). A variety of RT-PCR methods are known by the skilled person such as the methods described or referred in the Real Time PCR Special Issue of the METHODS Journal (Elsevier, December 2001, Volume 25, Issue 4. pages 383 to 481).

According to another embodiment of the present invention, when protein levels are assessed, antibodies which specifically recognize either DRD2 or Kir2.3 are produced. The antibodies are used alone or in combination. The level of binding of antibodies which specifically recognize the proteins will be higher in schizophrenic individuals than in normal individuals. An antibody against the Kir2.3. protein is described e.g. by Noam et al (1996)⁴⁶.

In order to normalize the level of binding to the total amount of proteins present in the tissue, the binding of the specific antibodies can be expressed relative to the binding of an antibody to a reference protein equally expressed in normal and diseased subjects.

In an alternate embodiment of the invention, the determination of the amount of modified and unmodified receptors can be made by isolation, e.g. partial isolation of the receptor protein or the potassium channel protein itself. Effective methods for isolation are gel electrophoresis, e.g. two dimensional gel electrophoresis, or isoelectric focusing techniques. A combination of these techniques with blotting techniques or other methods of immunological detection may provide an efficient tool for assessing the concentrations of quantities of the proteins in given samples. The isolated or partially purified proteins may be subjected to analysis of peptide fragments, obtained e.g. by proteolitic enzymes, which carry special properties e.g. may contain a site suitable for specific chemical modification.

According to an alternative embodiment the activity of the marker receptor protein DRD2 or the potassium channel Kir2.3 is measures as a value indicative of the level of said protein. Methods for determination of inward rectifier K+ currents and further methods to detect Kir2.3 are described e.g. in Noam et al (1996)⁴⁶ and Bannister et al. (1999)⁴⁷ and publications referred therein.

When the level of the marker protein or the marker gene is obtained and compared to the baseline level a certain threshold value can be determined above which the levels in samples from the diagnosed subjects are accepted as indicative of schizophrenia. This threshold value may depend on a variety of factors, including the measurement method, reliability of the baseline level, statistical (mathematical) considerations and the aim of the diagnosis (i.e. that the development of the disease has to be decided with certainty or only a susceptibility is to be ascertained) etc. As an extreme value, the baseline may coincide with the threshold value. Determination of levels above this threshold value may be indicative of the severity of the disease.

Advantageously, when a concrete method is optimized, a set of diseased subjects is also assayed to determine validity of the test by comparing results of the diseased sample to those of the normal sample.

Diagnostic kits are well known in the art. In particular, a number of diagnostic kits are available and are described in the prior art cited in the present description. Any of these kits can be used as analogue of the kits of the invention taking into account the specific features of the invention taught herein.

This project was supported by Hungarian Scientific Research Fund (OTKA-2001, T38373), Hungarian Ministry of Education (NKFP-2002 5/0079) and a bilateral grant between the Hungarian Prime Minister Office and Hungarian Academy of Sciences (4676/1/2003). L.G.P. was supported by János Bólyai scholarship of the Hungarian Ministry of Education.

### EXAMPLES

### Materials and Methods

Patients

Patients were recruited from the inpatient unit of the Department of Psychiatry and informed consent was obtained after explanation of the study. Each patient went through profound medical examination including physical and neurological examination and routine laboratory urine drug tests also. Each of them received the Mini International Neuropsychiatric Interview Plus (MINI Plus)²¹, and was diagnosed with schizophrenia or schizophreniform psychosis according to DSM-IV.²² Psychiatric symptoms were assessed using the Positive and Negative Syndrome Scale (PANSS)²³, the Clinical Global Impression (CGI) and the Global Assessment of Functioning (GAF) scale.²² Clinical ratings were performed by two trained psychiatrists. The study was conducted in accordance with Declaration of Helsinki. Demographical and medical data are detailed in (Table 2.) 7 female and 6 male drug-naive or drug-free schizophrenic patients and 10 healthy control individuals were analyzed.

Isolation of PBL and RNA from Schizophrenic Patients and Control Individuals

Lymphocytes fraction of 10 ml of total blood (collected in EDTA-treated tubes) was washed 3 times with red blood lysis buffer (5 mM MgCl₂, 10 mM NaCl, 10 mM Tris-HCl, pH 7.0, Promega, Madison, Wis., USA) to lyse erythrocytes. RNA isolation from clear pellet of lymphocytes was carried out by the RNA isolation kit of Macherey-Nagel (Macherey-Nagel, Düren, Germany) according the manufacturers' instruction. RNA samples were stored at −80° C. in the presence 30 U of Prime RNAse inhibitor (Eppendorf, Hamburg, Germany). The quality and quantity of isolated RNA was checked by electrophoresis and spectrophotometry (NanoDrop, Rockland, Del., USA) respectively.

Microarrays, Probe Preparations and Hybridizations

3200 amplified cDNA inserts from human lymphocyte cDNA Matchmaker library (Clontech, BD Biosciences Franklin Lakes, N.J., USA) (1200 clones) and 2000 sequence verified clones from mixed human library in pBluescript SK II (−) plasmid (New England Biolabs, Hertfordshire, England) were amplified by PCR with plasmid specific primers and purified with MultiScreen-PCR plate (Millipore, Billerica, Mass., USA), resuspended in 50% dimethyl sulfoxide/water, and arrayed on FMB cDNA slides (Full Moon BioSystems, Sunnyvale, Calif., USA) using a MicroGrid Total Array System (BioRobotics, Cambridge, UK) spotter. Post-processing and blocking of the microarrays were performed as described previously. For probe preparation, 2 μg of total RNA (a pool of 14 schizophrenic patients) was reverse transcribed using poly-dT primed Genisphere Expression Array 350 Detection Kit system (Genisphrere, Hatfield, P A, USA) according the manufacturer instruction. cDNA with capture sequence was hybridized onto human cDNA microarray containing 3200 human cDNA clones. Both the first step cDNA hybridization and the second step capture reagent hybridization were carried out in a Ventana hybridization station (Ventana Discovery, Tucson, Ariz., USA) by using the “antibody” protocol. First hybridization was performed at 40° C. for 6 hours in “Chiphyb” hybridization buffer (Ventana), then 2.5 μl of each Cy5 and Cy3 capture reagents were added to the slides in 200 μl “Ribohyb” hybridization buffer (Ventana) and incubated at 42° C. for 2 hours. After hybridization, the slides were washed in 0.2×SSC twice at RT for 10 min, then dried and scanned. Scanning and data analysis were done as describe earlier.²⁵

### Real-Time PCR

For QRT-PCR 1 μg of total RNA from individual schizophrenic patients and control individuals were reverse transcribed using SuperScript II RNase H⁻ Reverse transcriptase (Invitrogen, Carlsbad, Calif., USA). The reactions were carried out in 20 μl of final volume in the presence of 3.5 μM Random Hexamer Primer, 1× First-Strand Buffer (250 mM Tris-HCl (pH 8.3) 375 mM KCl, 15 mM MgCl₂), 500 nM dNTP mix (500 nM of each) 10 mM DTT, 40 U Prime RNAse inhibitor (Eppendorf) and 200 U of SuperScript II RNase H-Reverse transcriptase (Invitrogen) at 42° C. for 2 hours. RNA and random primer mixture was denatured at 65° C. for 5 minutes before RT reaction. The enzyme reaction was terminated by inactivating the reverse transcriptase at 70° C. for 15 minutes. The final volume of RT reaction was diluted 4 times. 1 μl of the diluted reaction mix was used for QRT-PCR using either SybrGreen dye or TaqMan probes. Reactions using SybrGreen protocol were done with ABsolute QPCR SYBR Green mix (ABGene, Surrey, UK) in RotorGene real-time Q-PCR machine (Corbett Research, Mortlake, Australia) according to manufacturer instruction at final concentration of 150 nM primers under following conditions; 15 min at 95° C., and 45 cycles of 95° C. for 15 sec, 60° C. for 25 sec and 72° C. for 25 sec. Fluorescence intensity of SybrGreen dye was detected after each amplification step. Melt temperature analysis was done after each reaction to check the quality of the reaction. PCR reactions with TaqMan probes were done using ABsolute QPCR mix (ABGene) according to manufacturer instruction in the presence of 300 nM of primers and 250 nM of TaqMan probes under the following conditions; 15 min at 95° C., and 45 cycles of 95° C. for 15 sec, 60° C. for 1 min. Intensity of fluorescein dye (FAM) was detected after each cycle. PCR primers and TaqMan probes were designed using the Primer Express Software (Applied Biosystems, Foster City, Calif., USA). Primers and TaqMan probes used in this study are summarized in Table 1. Dual-labeled probes were prepared by Bioneer (Daejeon, Korea) with Fluorescein dye at 5′ end and Dabsyl dye at 3′ end. The quality of the primers was verified by MS analysis provided by Bioneer. Non-template control sample was used for each PCR run to check the genomic DNA contaminations of cDNA template. Analysis of results was done using Pfaffl method.²⁶ Using this calculation method differences between the amplification efficiencies of reactions can be corrected.

TABLE 1

| Short | | | | Acc. |
|---|---|---|---|---|
| name | PCR Primers | TaqMan Probe | Gene Name | No. |
| DRD2 | For CTGCTCATCGCTGTCATCGT | TCGGCAACGTGCTG | Human D2 | X51362 |
| | Rev CTCGCGGGACACAGCC | GTGTGCA | dopamine receptor | |
| Kir2.3 | For CTTCCCCCACTGACTCTTCAAG | TGCCCTCTTTGCTCT | Homo sapiens | NM_004981 |
| | Rev CCAGCAGTCCAGCCACCTT | CAGAACCTTGG | potassium inwardly- | |
| | | | rectifying channel | |
| | | | (Kir2.3) | |
| Actin | For TCACCGAGCGCGGCT | CAGCTTCACCACCAC | Human actin, beta | NM_001101 |
| | Rev TAATGTCACGCACGATTTCCC | GGCCGA | (ACTB), | |
| HPRT | For TGACACTGGCAAAACAATGCA | CTTTGCTTTCCTTGG | Human | M31642 |
| | Rev GCTTGCGACCTTGACCATCT | TCAGGCAGTATAATC | hypoxanthine | |
| | | CA | phosphoribosyl- | |
| | | | transferase (HPRT) | |

### Statistical Analysis

Unpaired Student's t test (two-tailed) was applied to determine the p value. To divide the gene expression dataset into groups of observations that are similar to each other, agglomerative hierarchical clustering was applied. The employed method was the group average method. Euclidean metric (root sum-of-squares of differences) was used to calculate the dissimilarities between observations. The routine is implemented in the R1.8.1 statistical software.

Results

Patients and Control Individuals

We screened 13 drug-naive or drug-free schizophrenic patients (7 female and 6 male, age range 23-67 years, average 34±12) and 10 control individuals (5 male and 5 female, age range 28-65 years, average 38±10) to identify novel peripheral genetic markers of schizophrenia. Demographic data of schizophrenic patients and control individuals are summarized in Table 2. Neither affected nor non-affected individuals were under antipsychotic treatment or other medication.

TABLE 2

| | | | | | | | | | | Medication on | | |
|---|---|---|---|---|---|---|---|---|---|---|---|---|
| | Status (duration of | | Duration | PANSS | | | | | | date of sampling | | |
| Patients | drug-free period) | Age | of illness | positive | negative | global | Σ | CGI | GAF | APS | ANX | other |
| Male 2 | F (1 month) | 34 | 3 years | 26 | 25 | 55 | 106 | 4 | 50 | Ø | Ø | Ø |
| Male 3 | N | 34 | 6 months | 25 | 21 | 36 | 82 | 4 | 40 | Ø | Ø | Ø |
| Male 4 | F (4 months) | 36 | 4 years | 33 | 31 | 65 | 129 | 6 | 25 | Ø | Ø | Ø |
| Male 6 | N | 25 | 4.5 years | 29 | 36 | 66 | 131 | 6 | 20 | Ø | Ø | Ø |
| Male 7 | N | 25 | 7 months | 34 | 32 | 74 | 140 | 6 | 20 | Ø | Ø | Ø |
| Male 8 | N | 24 | 6 months | 14 | 37 | 49 | 100 | 5 | 25 | Ø | Ø | Ø |
| Female 1 | F (3 months) | 46 | 15 years | 31 | 25 | 58 | 114 | 6 | 30 | Ø | Ø | Ø |
| Female 2 | F (3 months) | 34 | 14 years | 34 | 40 | 85 | 159 | 7 | 15 | Ø | Ø | Ø |
| Female 3 | F (1 year) | 67 | 17 years | 29 | 28 | 51 | 108 | 5 | 30 | Ø | Ø | Ø |
| Female 4 | F (1 year) | 29 | 5 years | 29 | 30 | 54 | 113 | 6 | 30 | Ø | Ø | Ø |
| Female 5 | N | 27 | 9 months | 40 | 34 | 81 | 155 | 7 | 15 | Ø | Ø | Ø |
| Female 6 | N | 33 | 8 months | 22 | 23 | 54 | 99 | 4 | 40 | Ø | Ø | Ø |
| Female 8 | N | 23 | 3 months | 22 | 28 | 62 | 112 | 5 | 30 | Ø | Ø | Ø |

N: drug-naive, F: drug-free
PANSS: Positive and Negative Syndrome Scale
positive: positive syndrome items
negative: negative syndrome items
global: global syndrome items
Σ: PANSS total score
CGI: Clinical Global Impression
GAF: Global Assessment of Functioning
APS: antipsychotic
ANX: anxiolytic

Pre-Screen by cDNA Microarray Analysis

cDNA microarray analysis was performed in order to pre-screen expression pattern of schizophrenic PBL and to identify potential candidate peripheral marker genes. Genes with altered expression were subjected to further analysis using QRT-PCR with SybrGreen dye and dual labeled TaqMan probes. Total RNA pool of 13 affected male and female patients and 10 control individuals were reverse transcribed and hybridized onto human cDNA microarray containing 3200 human cDNA clones. Positive clones showing more than two-fold change in their expression compared to controls were sequenced. Among others, 2.56±0.26- and 6.75±2.3-fold increase in expression level of DRD2 and Kir2.3 was detected, respectively, in schizophrenic patients compared to healthy individuals (FIG. 2). Based on this preliminary data and considering the potential role of these genes in pathogenesis of schizophrenia, expression levels of DRD2 and Kir2.3 genes were subjected to further analysis.

Reference Controls and Baseline Values

After reverse transcription of 13 individual total RNA samples of affected and 10 non-affected individuals, QRT-PCR was performed on cDNA templates mixed with primers and TaqMan probes specific for DRD2 and Kir2.3 genes. β-actin or hypoxanthine phosphoribosyl-transferase (HPRT) genes were used as internal reference controls. Relative quantification using Ct values and reaction efficiency values was performed to determine the exact ratio between the normalized Ct values of both genes in all individual affected samples compared to the average values calculated from Ct values of the 5-5 sex matched individual control samples.

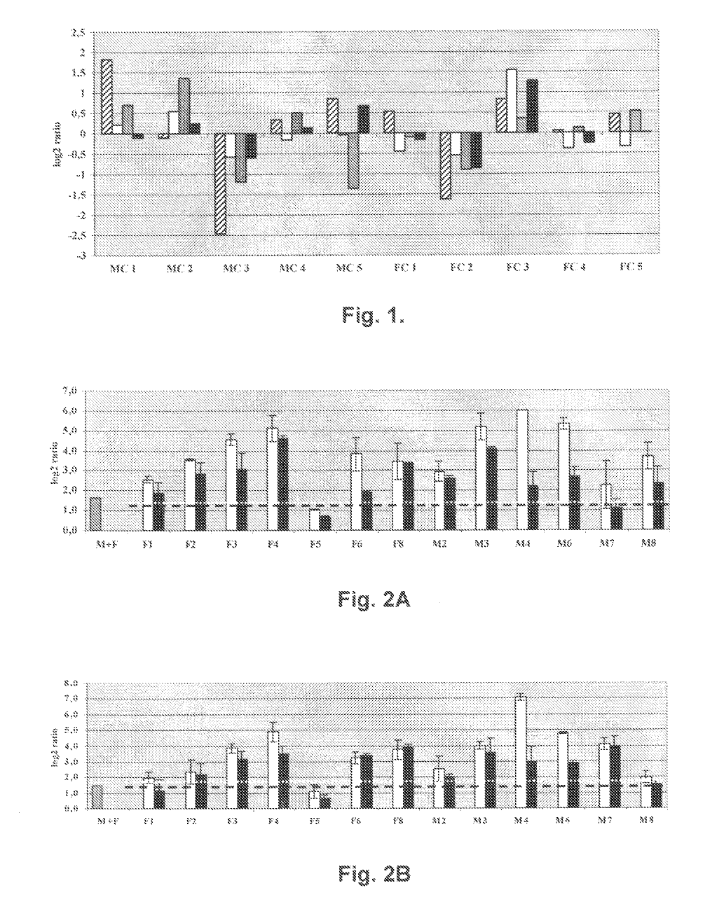

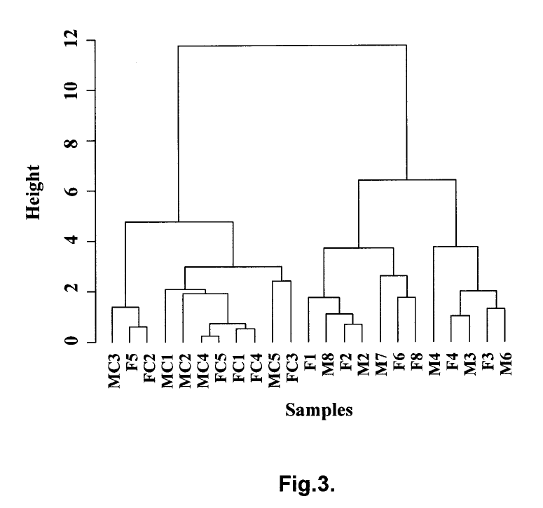

To determine the variation of DRD2 and Kir2.3 mRNA levels in the non-affected samples, relative expression levels of both genes were calculated. The variation of relative expression level of DRD2 and Kir2.3 genes in control samples is shown in FIG. 1. Relative mRNA level of DRD2 and Kir2.3 in non-affected samples were changed in a range of 0.39- to 2.56-fold and 0.18- to 3.54-fold, respectively, using the SybrGreen method and 0.55- and 2.42-fold and 0.67- to 2.92-fold, respectively, using TaqMan probe method. Considering these variations of relative expression level of DRD2 and Kir2.3 genes in non-affected control samples, a background level of relative expression was calculated. Only affected PBLs with relative expression level of DRD2 and Kir2.3 higher than any of the non-affected control were regarded as positives. Using SybrGreen method, ratios above 2.56 and 3.54 were regarded as up-regulated in the case of DRD2 and Kir2.3 genes, respectively. In the case of TaqMan protocol, ratios above 2.42 and 2.92 were regarded as up-regulated in the case of DRD2 and Kir2.3 genes, respectively (FIG. 2, white dotted and black dashed lines).

Overexpression of the DRD2 Gene

DRD2 gene was found to be significantly overexpressed in 6 male and 6 female schizophrenic patients compared to healthy controls in a range of 5.7-fold to 64.7-fold using the SybrGreen method. Relative expression level of DRD2 gene of one female affected PBL (F5) was under the background level. In this case a 2.1-fold overexpression could be detected. The average increase of DRD2 gene expression detected by the SybrGreen method was 21.4±18.5-fold (p=0.0019) (FIG. 2, A, white bars). Using TaqMan probe, 6 female and 5 male schizophrenic PBLs showed increased mRNA level of DRD2 gene (3.7- to 24.1-fold). Relative expression levels of DRD2 of one female and one male affected PBLs (F5, M7) were under the calculated background level (1.6- and 2.2-fold, respectively). The average increase of DRD2 gene expression analyzed by TaqMan protocol was 7.8±6.3-fold (p=0.0023) (FIG. 2A, black bars).

Overexpression of the Kir2.3 Gene

In the case of 6 male and 6 female schizophrenic PBL, the expression level of Kir2.3 gene was also above the background expression level compared to healthy controls. Ratios of mRNA level of Kir2.3 in affected versus non-affected sample were varied in a range of 4- to 133.6-fold using the SybrGreen method. Relative expression level of Kir2.3 gene of one female affected PBL (F5) was under the background value (2.2-fold). The average increase of relative expression level of Kir2.3 analyzed by SybrGreen method was 22±34.7-fold (p=0.0535) (FIG. 2B, white bars). These changes in expression level were confirmed by the method using dual labeled TaqMan probes as well. 5 female and 6 male schizophrenic PBL showed increased mRNA level of Kir2.3 gene (2.8- to 15.8-fold). In case of 2 female affected PBLs (F1, F5), relative expression levels of Kir2.3 were under the calculated background level (2.4- and 1.6-fold, respectively). The average increase of Kir2.3 gene expression analyzed by TaqMan probe was 8.2±4.8-fold (p=0.0001) (FIG. 2B, black bars). Differences in expression levels observed by the two different real-time PCR methods could be explained by the more precise and sensitive features of the TaqMan protocol.

Similarity Groups

To determine similarity groups of gene expression dataset, agglomerative hierarchical clustering was applied. The non-affected control and affected samples were correctly classified into two separate clusters with the evident exception of the schizophrenic sample F5. There was no significant differences between relative expression levels of female and male patients (FIG. 3).

Among the genes that were found to be differently expressed in affected PBL by microarray method, up-regulation in expression of two genes, DRD2 and Kir2.3 were confirmed by SybrGreen and TaqMan probes based QRT-PCR. Since neither affected nor non-affected individuals were under antipsychotic or other medication treatment, elevated mRNA levels of DRD2 and Kir2.3 reflect the disorder itself, not the effect of medication. Thus, DRD2 and Kir2.3 can serve as additional peripheral genetic markers for prediction of schizophrenia. High-throughput analysis of already reported molecular markers and genes in combination with the presented ones help the earlier and more precise diagnosis of this disorder. Diagnostic tests based on these results can be developed and help manage schizophrenia from the beginning to the chronic state.

A number of methods useful in the present invention is described in the prior art cited herein which are incorporated herein by reference.

### REFERENCES

1. McKenna F, McLaughlin P J, Lewis B J, Sibbring G C, Cummerson J A, Bowen-Jones D et al. Dopamine receptor expression on human T- and B-lymphocytes, monocytes, neutrophils, eosinophils and NK cells: a flow cytometric study. J Neuroimmunol 2002; 132: 34-40.
2. Ricci A, Bronzetti E, Felici L, Greco S, Amenta F. Labeling of dopamine D3 and D4 receptor subtypes in human peripheral blood lymphocytes with [3H]7-OH-DPAT: a combined radioligand binding assay and immunochemical study. J Neuroimmunol 1998; 92: 191-5.
3. Ilani T, Ben-Shachar D, Strous R D, Mazor M, Sheinkman A, Kotler M et a/. A peripheral marker for schizophrenia: Increased levels of D3 dopamine receptor mRNA in blood lymphocytes. Proc Natl Acad Sci USA 2001; 98: 625-228.
4. Ricci A, Bronzetti E, Felici L, Tayebati S K, Amenta F. Dopamine D4 receptor in human peripheral blood lymphocytes: a radioligand binding assay study. Neurosci Lett 1997; 229: 130-134.
5. Lewis D A, Lieberman J A. Catching up on schizophrenia: natural history and neurobiology. Neuron 2000; 28:325-34.
6. Abi-Dargham A, Rodenhiser J, Printz D, Zea-Ponce Y, Gil R, Kegeles L S. et al. Increased baseline occupancy of D2 receptors by dopamine in schizophrenia. Proc Natl Acad Sci USA 2000; 97: 8104-9.
7. Seeman P. Dopamine receptors and the dopamine hypothesis of schizophrenia. Synapse 1987; 1: 133-52.
8. Ricci A, Veglio F and Amenta F. Radioligand binding characterization of putative dopamine D3 receptor in human peripheral blood lymphocytes with [3H] 7-OH-DPAT. J. Neuroimmunol 1995; 58: 139-144.
9. Bondy B, de Jonge S, Pander S, Primbs J, Ackenheil M. Identification of dopamine D4 receptor mRNA in circulating human lymphocytes using nested polymerase chain reaction. J Neuroimmunol 1996; 71: 139-144.
10. Bishopric N H, Cohen H J, Lefkowitz R J. Beta adrenergic receptors in lymphocyte subpopulations. J Allergy Clin Immunol 1980; 65: 29-33.
11. Kwak Y T, Koo M S, Choi C H, Sunwoo I. Change of dopamine receptor mRNA expression in lymphocyte of schizophrenic patients. BMC Med Genet 2001; 2:3.
12. Perl O, llani T, Strous R D, Lapidus R, Fuchs S. The alpha7 nicotinic acetylcholine receptor in schizophrenia: decreased mRNA levels in peripheral blood lymphocytes. FASEB J2003; 17: 1948-50.
13. Levite M, Chowers Y, Ganor Y, Besser M, Hershkovits R, Cahalon L. Dopamine interacts directly with its D3 and D2 receptors on normal human T cells, and activates betal integrin function. Eur J Immunol 2001; 12: 3504-12.
14. Barbanti P, Fabbrini G, Ricci A, Bruno G, Cerbo R, Bronzetti E, Amenta F, Luigi Lenzi G. Reduced density of dopamine D2-like receptors on peripheral blood lymphocytes in Alzheimer's disease. Mech Ageing Dev 2000; 120: 65-75.
15. Mimics K, Middleton F A, Marquez A, Lewis D A, Levitt P. Molecular characterization of schizophrenia viewed by microarray analysis of gene expression in prefrontal cortex. Neuron 2000; 28: 53-67.
16. Mimics K, Middleton F A, Lewis D A, Levitt P. Analysis of complex brain disorders with gene expression microarrays: schizophrenia as a disease of the synapse. Trends Neurosci 2001; 24: 479-86.
17. Mimics K, Middleton F A, Stanwood G D, Lewis D A, Levitt P. Disease-specific changes in regulator of G-protein signaling 4 (RGS4) expression in schizophrenia. Mol Psychiatry 2001; 6: 293-301.
18. Middleton F A, Mimics K, Pierri J N, Lewis D A, Levitt P. Gene expression profiling reveals alterations of specific metabolic pathways in schizophrenia. J Neurosci 2002; 22: 2718-29.
19. Hakak Y, Walker J R, Li C, Wong W H, Davis K L, Buxbaum J D, Haroutunian V, Fienberg M. Genome-wide expression analysis reveals dysregulation of myelination-related genes in chronic schizophrenia. Proc Natl Acad Sci USA 2001; 98: 4746-51.
20. Marcotte E R, Srivastava L K, Quirion R. cDNA microarray and proteomic approaches in the study of brain diseases: focus on schizophrenia and Alzheimer's disease. Pharmacol Ther 2003; 100: 63-74.
21. Balázs J, Bitter I. [Study on construct validity of the M.I.N.I. PLUS interview]. Psychiat Hung 2000; 15: 134-44.
22. American Psychiatric Association. DSM-I: Diagnostic and Statistical Manual of Mental Disorders, 4^{th} ed. Washington, D.C.: American Psychiatric Associaton, 1994.
23. Kay S R, Fiszbein A, Opler L A. The positive and negative syndrome scale (PANSS) for schizophrenia. Schizophr Bull. 1987; 13: 261-76.
24. Puskas L G, Hackler L Jr, Kovacs G, Kupihar Z, Zvara A, Micsik T, van Hummelen P. Recovery of cyanine-dye nucleotide triphosphates. Anal Biochem 2002; 305: 279-81.
25. Onody A, Zvara A, Hackler L J, Vigh L, Ferdinandy P, Puskas L G. Effect of classic preconditioning on the gene expression pattern of rat hearts: a DNA microarray study. FEBS Lett. 2003; 536: 35-40.
26. Pfaffl MW. A new mathematical model for relative quantification in real-time RT-PCR. Nucleic Acids Res 2001; 29: e45.
27. Albert P R, Neve K A, Bunzow J R, Civelli O. Coupling of cloned rat dopamine-D2 receptor to inhibition of adenylyl cyclase and prolactin secretion. J Biochem 1990; 265, 2098-2104.
28. Myeong H, Jeoung D, Kim H, Ha J H, Lee Y, Kim K H et al. Genomic analysis and functional expression of canine dopamine D2 receptor. Gene 2000; 257: 99-107.
29. Cohen N A, Sha Q, Makhina E N, Lopatin A N, Linder M E, Snyder S H, Nichols C G. Inhibition of an inward rectifier potassium channel (Kir2.3) by G-protein betagamma subunits. J Biol Chem 1996; 271: 32301-5.
30. Perier F, Radeke C M, Vandenberg C A. Primary structure and characterization of a small-conductance inwardly rectifying potassium channel from human hippocampus. Proc Natl Acad Sci USA 1994; 91: 6240-4.
31. Uchida S, Akaike N, Nabekura J. Dopamine activates inward rectifier K+ channel in acutely dissociated rat substantia nigra neurones. Neuropharmacology2000; 39: 191-201.
32. Lavine N, Ethier N, Oak J N, Pei L, Liu F, Trieu P et al. G protein-coupled receptors form stable complexes with inwardly rectifying potassium channels and adenylyl cyclase. J Biol Chem 2002; 277: 46010-9.
33. Roberts D A, Balderson D, Pickering-Brown S M, Deakin J F, Owen F. The abundance of mRNA for dopamine D2 receptor isoforms in brain tissue from controls and schizophrenics. Brain Res Mol Brain Res 1994; 25: 173-175.
34. Tallerico T, Novak G, Liu I S, Ulpian C, Seeman P. Schizophrenia: elevated mRNA for dopamine D2 (Longer) receptors in frontal cortex. Brain Res Mol Brain Res 2001; 87: 160-165.
35. Vawter M P, Ferran E, Galke B, Cooper K, Bunney W E, Byerley W. Microarray screening of lymphocyte gene expression differences in a multiplex schizophrenia pedigree. Schizophr Res 2004; 67: 41-52.
36. Strausberg, R. L. et al, Generation and initial analysis of more than 15,000 full-length human and mouse cDNA sequences, Proc. Natl. Acad. Sci. U.S.A. 99 (26), 16899-16903 (2002)
37. Araki, K., Kuwano, R., Morii, K., Hayashi, S., Minoshima, S., Shimizu, N., Katagiri, T., Usui, H., Kumanishi, T. and Takahashi, Y., Structure and expression of human and rat D2 dopamine receptor genes, Neurochem. Int. 21 (1), 91-98 (1992)
38. Dearry, A., Falardeau, P., Shores, C. and Caron, M. G., D2 dopamine receptors in the human retina: cloning of cDNA and localization of mRNA, Cell. Mol. Neurobiol. 11 (5), 437-453 (1991)
39. Monsma, F. J. Jr., McVittie, L. D., Gerfen, C. R., Mahan, L. C. and Sibley, D. R. TITLE Multiple D2 dopamine receptors produced by alternative RNA splicing JOURNAL Nature 342 (6252), 926-929 (1989)
40. Dal Toso, R., Sommer, B., Ewert, M., Herb, A., Pritchett, D. B., Bach, A., Shivers, B. D. and Seeburg, P. H., The dopamine D2 receptor: two molecular forms generated by alternative splicing, EMBO J. 8 (13), 4025-4034 (1989)
41. Grandy, D. K., Marchionni, M. A., Makam, H., Stofko, R. E., Alfano, M., Frothingham, L., Fischer, J. B., Burke-Howie, K. J., Bunzow, J. R., Server, A. C. et al., Cloning of the cDNA and gene for a human D2 dopamine receptor, Proc. Natl. Acad. Sci. U.S.A. 86 (24), 9762-9766 (1989)
42. Perier, F., Radeke, C. M. and Vandenberg, C. A., Primary structure and characterization of a small-conductance inwardly rectifying potassium channel from human hippocampus, Proc. Natl. Acad. Sci. U.S.A. 91 (13), 6240-6244 (1994)
43. Tang, W. and Yang, X. C., Cloning a novel human brain inward rectifier potassium channel and its functional expression in Xenopus oocytes, FEBS Lett. 348 (3), 239-243 (1994)
44. Makhina, E. N., Kelly, A. J., Lopatin, A. N., Mercer, R. W. and Nichols, C. G., Cloning and expression of a novel human brain inward rectifier potassium channel, J. Biol. Chem. 269 (32), 20468-20474 (1994)
45. Budarf, M. L., Perier, F., Barnoski, B. L., Bell, C. J. and Vandenberg, C. A., Assignment of the human hippocampal inward rectifier potassium channel (HIR) gene to 22q13.1, Genomics 26 (3), 625-629 (1995)
46. Noam A. Cohen, Qun Shan, Elena N. Makhina, Anatoli N. Lopatin, Maurine E. Linder, Solomon H. Snyder and Collin, G. Nichols, Inhibition of an Inward Rectifier Potassium Channel (Kir2.3) by G-protein^{βγ}Subunits, J. Biol. Chem. 271 (50), 32301-32305 (1996)
47. Bannister J P, Young B A, Sivaprasadarao A, Wray D., Conserved extracellular cysteine residues in the inwardly rec potassium channel Kir2.3 are required for function but not expression in the membrane, FEBS Lett., 24, 458 83): 393-9, (1999).

1. A method for diagnosing schizophrenia in a subject comprising
assessing the expression level of at least a gene encoding Kir2.3 (inwardly rectifying potassium channel 2.3) in a biological sample taken from said subject and
comparing the expression level in the biological sample to a baseline expression level of the respective gene typical of healthy subjects,
wherein a higher gene expression level in the biological sample is considered as an indication of the fact that the subject suffers in or is susceptible to schizophrenia.

2-16. (canceled)

17. The method of claim 1 further comprising assessing the expression level of a further gene encoding DRD2 (dopamine D2 receptor) in the biological sample and comparing the expression level of said further gene in the biological sample to a baseline expression level of the respective further gene typical of healthy subjects.

18. The method of claim 17 wherein assessment of the expression level comprises
hybridization of one or more hybridization probe to one or more nucleic acid, preferably an mRNA obtained from the biological sample, said hybridization probe or said nucleic acid having a sequence
of a gene encoding Kir2.3,
if the level of DRD2 is also assessed, of a gene encoding DRD2,
of a part thereof or
of a sequence complementer to any of the above; and
detection of the hybridization by a means for producing a detectable signal upon specific binding of the hybridization probe.

19. The method of claim 18 wherein the expression level is determined by microarray analysis or a PCR method, preferably a real time PCR method.

20. The method of claim 18, wherein the hybridization probe is a probe comprising an oligonucleotide specifically hybridizing to any of the nucleic acids or a complementer or a part thereof.

21. The method of claim 18, wherein the means for producing a detectable signal comprises a label.

22. The method of claim 21, wherein the label is an enzymatic label, a fluorescent label or a radioactive label.

23. The method of claim 21, wherein the probe comprises an oligonucleotide and a label bound thereto, and/or the probe comprises a fluorescent label and a quencher, and/or the label is a fluorescent label, and/or the probe is an oligonucleotide and the label is an intercalating agent, e.g. SYBR-green or ethidium bromide.

24. The method of claim 1, wherein the biological sample is taken from brain, spinal cord, lymphatic fluid, lymphatic organ, bone marrow, blood, and/or the biological sample comprises immune cells, peripheral blood cells, peripheral blood lymphocites and the gene expression level is assessed in these cells.

25. The method of claim 24, wherein the biological sample comprises peripheral blood lymphocites and the gene expression level is assessed in these cells.

26. A method for diagnosing schizophrenia in a subject comprising
assessing the expression level of at least a gene encoding DRD2 (dopamine D2 receptor) in peripheral blood lymphocytes in a biological sample taken from said subject and
comparing the expression level in the biological sample to a baseline expression level of the respective gene typical of healthy subjects,
wherein a higher gene expression level in the biological sample is considered as an indication of the fact that the subject suffers in or is susceptible to schizophrenia.

27. The method of claim 26 further comprising assessing the expression level of a further gene encoding Kir2.3 (inwardly rectifying potassium channel 2.3) in the biological sample and the expression level of said further gene in the biological sample is compared to a baseline expression level of the respective further gene typical of healthy subjects.

28. The method of claim 27 wherein assessment of the expression level comprises
hybridization of one or more hybridization probe to one or more nucleic acid, preferably an mRNA obtained from the biological sample, said hybridization probe or said nucleic acid having a sequence
of a gene encoding DRD2,
if the level of Kir2.3 is also assessed, of a gene encoding Kir2.3,
of a part thereof or
of a sequence complementer to any of the above; and
detection of the hybridization by a means for producing a detectable signal upon specific binding of the hybridization probe.

29. The method of claim 28 wherein the expression level is determined by microarray analysis, preferably by a PCR method, preferably a real time PCR method.

30. The method of claim 28, wherein the hybridization probe is a probe comprising an oligonucleotide specifically hybridizing to any of the nucleic acids or a complementer or a part thereof.

31. The method of claim 28, wherein the means for producing a detectable signal comprises a label.

32. The method of claim 31, wherein the label is an enzymatic label, a fluorescent label or a radioactive label.

33. The method of claim 31, wherein the probe comprises an oligonucleotide and a label bound thereto, and/or the probe comprises a fluorescent label and a quencher, and/or the label is a fluorescent label, and/or the probe is an oligonucleotide and the label is an intercalating agent, e.g. SYBR-green or ethidium bromide.
